(19) United States
(12) Patent Application Publication
(10) Pub. No.: US 2018/0318556 A1
(43) Pub. Date: Nov. 8, 2018

(54) SYSTEM, METHOD, AND APPARATUS FOR SELECTIVELY ACCESSING AN INTERIOR LUMEN OF A PATIENT VESSEL

(71) Applicant: THE GENERAL HOSPITAL CORPORATION, Boston, MA (US)

(72) Inventors: Gabriel GRUIONU, Boston, MA (US); Jarone LEE, Boston, MA (US); Lucian GRUIONU, Boston, MA (US)

(21) Appl. No.: 15/772,972

(22) PCT Filed: Nov. 3, 2016
(86) PCT No.: PCT/US2016/060293

§ 371 Date: May 2, 2018

Related U.S. Application Data
(60) Provisional application No. 62/250,474, filed on Nov. 3, 2015.

(51) Int. Cl. A61M 25/06

(52) U.S. Cl. CPC A61M 25/0606; A61M 29/00; A61M 25/0625

(57) ABSTRACT

Systems, methods, and apparatuses for selectively accessing an interior lumen of a patient vessel include a needle. The needle has a needle body and a needle distal end. A first catheter has a first catheter body, defining a first catheter inner lumen, and a first catheter distal end. The first catheter inner lumen is larger in diameter than the needle body and the needle distal end. The first catheter distal end has a first catheter open tip. A second catheter has a second catheter body, defining a second catheter inner lumen, and a second catheter distal end. The second catheter inner lumen is larger in diameter than the first catheter body and the first catheter distal end. The second catheter distal end has a second catheter open tip. At least two of the needle, the first catheter, and the second catheter are selectively joined concentrically together.

### RELATED APPLICATION

This application claims priority from U.S. Provisional Application No. 62/250,474, filed 3 Nov. 2015, the subject matter of which is incorporated herein by reference in its entirety.

### TECHNICAL FIELD

This disclosure relates to a system, apparatus, and method for selectively accessing an interior lumen of a patient vessel and, more particularly, to a method for selectively accessing an interior lumen of a patient vessel.

### BACKGROUND

Peripheral Intravenous (PIV) cannulation is a common hospital procedure in which a sharp needle and a plastic access tube (a cannula) is inserted into the vein of a patient to gain vascular access for subsequent administration of fluids, medication, and/or nutrients. PIV cannulation is particularly difficult to perform in critically ill, hypotensive (low blood pressure) patients who may have severe blood loss as a result of many causes including, but not limited to, traumatic hemorrhagic shock and septic shock. These patients may require rapid infusion of a large volume of fluids and/or blood products.

Occasionally, certain procedures include using a large bore catheter, such as, but not limited to, a 14G catheter, to infuse fluid and/or blood products into a patient at a quicker rate than what a small bore catheter, such as, but not limited to, a 22G catheter, would allow. However, in procedures where it may be beneficial to use large bore catheters, some patients, such as patients with hypotension, or any other similar complicating conditions, may have difficulty with blood vessel access with large bore catheters. Instead, small bore catheters may be more desirable to use with certain patients.

### SUMMARY

In an aspect, a system for selectively accessing an interior lumen of a patient vessel is provided. The system for selectively accessing an interior lumen of a patient vessel includes a needle. The needle has an elongate needle body and a needle distal end. The system for selectively accessing an interior lumen of a patient vessel includes a first catheter. The first catheter has an elongate first catheter body and a first catheter distal end. The first catheter body defines a first catheter inner lumen. The first catheter inner lumen is larger in diameter than the needle body and the needle distal end. The first catheter distal end has a first catheter open tip. The system for selectively accessing an interior lumen of a patient vessel includes a second catheter. The second catheter has an elongate second catheter body and a second catheter distal end. The second catheter body defines a second catheter inner lumen. The second catheter inner lumen is larger in diameter than the first catheter body and the first catheter distal end. The second catheter distal end has a second catheter open tip. The second catheter is capable of being attached to a fluid source. At least two of the needle, the first catheter, and the second catheter are selectively joined together to be concentrically arranged relative to one another.

In another aspect, a method for selectively accessing an interior lumen of a patient vessel is provided. A system for selectively accessing an interior lumen of a patient vessel is provided. The system for selectively accessing an interior lumen of a patient vessel includes a needle. The needle has an elongate needle body and a needle distal end. The system for selectively accessing an interior lumen of a patient vessel includes a first catheter. The first catheter has an elongate first catheter body and a first catheter distal end. The first catheter body defines a first catheter inner lumen. The first catheter inner lumen is larger in diameter than the needle body and the needle distal end. The first catheter distal end has a first catheter open tip. The system for selectively accessing an interior lumen of a patient vessel includes a second catheter. The second catheter has an elongate second catheter body and a second catheter distal end. The second catheter body defines a second catheter inner lumen. The second catheter inner lumen is larger in diameter than the first catheter body and the first catheter distal end. The second catheter distal end has a second catheter open tip. The second catheter is capable of being attached to a fluid source. The needle is inserted into the first catheter inner lumen, with the needle and the first catheter being concentrically arranged relative to one another. The first catheter, with the inserted needle, is inserted into the second catheter inner lumen, with the first catheter, the needle, and the second catheter concentrically arranged relative to one another. The first catheter, the second catheter, and the needle are collectively inserted into a patient vessel lumen. At least one of the needle and the first catheter are removed from the second catheter inner lumen, while maintaining the second catheter in the patient vessel lumen.

In another aspect, a concentric catheter device is provided. The concentric catheter device includes a needle. The needle has a needle proximal end and a needle distal end. The needle proximal end and the needle distal end are longitudinally spaced apart by an elongate needle body. The needle has a needle outer wall. The needle distal end has a needle tip. The concentric catheter device includes a first catheter. The first catheter has a first catheter proximal end and a first catheter distal end. The first catheter proximal end and the first catheter distal end are longitudinally spaced apart by an elongate first catheter body. The first catheter has a first catheter outer wall. The first catheter has a first catheter inner lumen. The first catheter distal end has a first catheter open tip. The first catheter inner lumen extends between the first catheter proximal end and the first catheter open tip. The first catheter inner lumen is larger in diameter than the needle outer wall. The first catheter proximal end is capable of being attached to a fluid source. The concentric catheter device includes a second catheter. The second catheter has a second catheter proximal end and a second catheter distal end. The second catheter proximal end and the second catheter distal end are longitudinally spaced apart by an elongate second catheter body. The second catheter has a second catheter outer wall. The second catheter has a second catheter inner lumen. The second catheter distal end has a second catheter open tip. The second catheter inner lumen extends between the second catheter proximal end and the second catheter open tip. The second catheter inner lumen is larger in diameter than the first catheter outer wall. The second catheter proximal end is capable of being attached to a fluid source. When the second catheter is joined to the first catheter, the first catheter open tip and at least a portion of the first catheter distal end longitudinally extend distally from the second catheter open tip. When the needle is joined to the first catheter, the needle tip and at least a portion of the needle distal end longitudinally extend distally from the first catheter open tip.

### BRIEF DESCRIPTION OF THE DRAWINGS

For a better understanding, reference may be made to the accompanying drawings, in which:

FIG. 1 is a side view of a concentric catheter device according to one aspect of the present invention; and

FIG. 2 is a side view of an element of the aspect of FIG. 1;

FIG. 3 is a side view of an element of the aspect of FIG. 1;

FIG. 4 is a side view of an element of the aspect of FIG. 1;

FIG. 5 is a side view of an element of the aspect of FIG. 1;

FIG. 6 is a side view of an element of the aspect of FIG. 1;

FIG. 7 is a side view of an element of the aspect of FIG. 1;

FIG. 8 is a side view of an element of the aspect of FIG. 1;

FIGS. 9-13 illustrate an example sequence of operation of a portion of the aspect of FIG. 1;

FIGS. 14-18 illustrate an example sequence of operation of a portion of the aspect of FIG. 1;

FIGS. 19-21 illustrate an example sequence of operation of a portion of the aspect of FIG. 1;

FIGS. 22-26 illustrate an example sequence of operation of a portion of the aspect of FIG. 1;

FIGS. 27-32 illustrate an example sequence of operation of a portion of the aspect of FIG. 1.

FIG. 33 is a side view of an element of the aspect of FIG. 1;

FIG. 34 is a side view of an element of the aspect of FIG. 1;

FIG. 35-39 illustrate an example sequence of operation of a portion of the aspect of FIGS. 33-34;

FIG. 40 is a partial cross-sectional view of an element of the aspect of FIG. 33; and

FIG. 41 is a partial cross-sectional view of an example of the aspect of FIG. 33.

### DESCRIPTION OF EMBODIMENTS

Unless defined otherwise, all technical and scientific terms used herein have the same meaning as is commonly understood by one of skill in the art to which the present disclosure pertains.

As used herein, the term “patient” can refer to any warm-blooded organism including, but not limited to, human beings, pigs, rats, mice, dogs, goats, sheep, horses, monkeys, apes, rabbits, cattle, farm animals, livestock, etc.

As used herein, the term “user” can be used interchangeably to refer to an individual who prepares for, assists, and/or performs a procedure.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” and “between about X and Y” can be interpreted to include X and Y.

As used herein, phrases such as “between about X and Y” can mean “between about X and about Y.”

As used herein, phrases such as “from about X to Y” can mean “from about X to about Y.”

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “directly adjacent” another feature may have portions that overlap or underlie the adjacent feature, whereas a structure or feature that is disposed “adjacent” another feature may not have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the Figures. It will be understood that the spatially relative terms can encompass different orientations of a device in use or operation, in addition to the orientation depicted in the Figures. For example, if a device in the Figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or Figures unless specifically indicated otherwise.

The invention comprises, consists of, or consists essentially of the following features, in any combination.

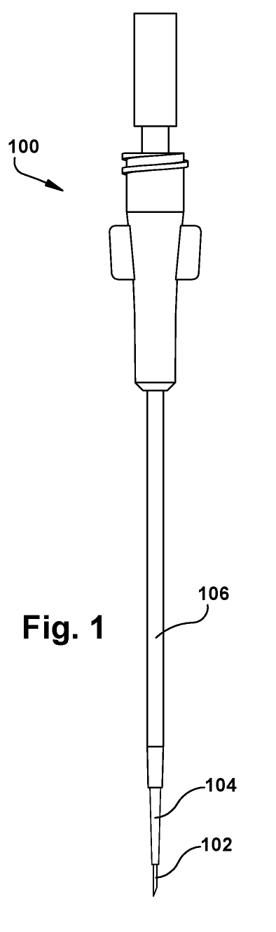

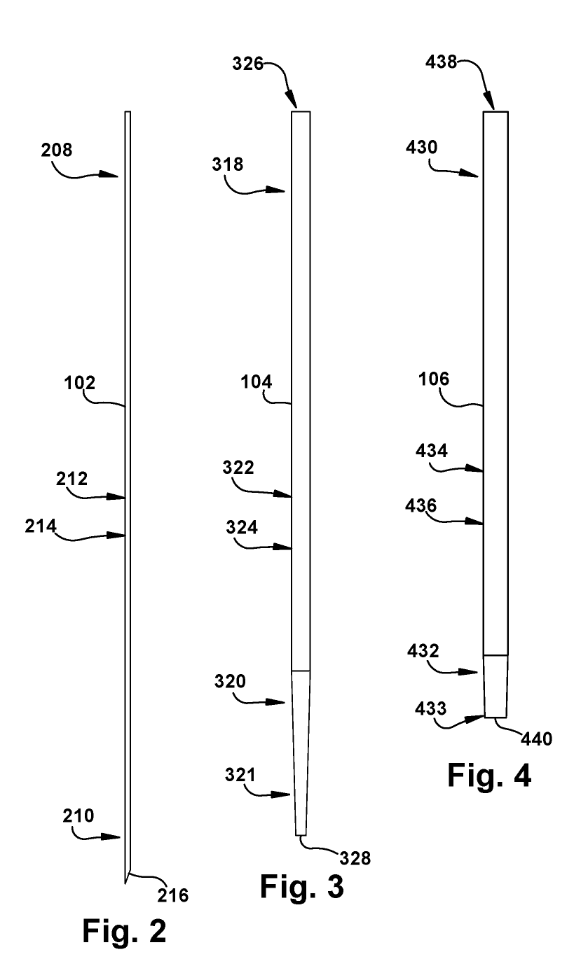

FIG. 1 depicts a concentric catheter device 100. The term “concentric” is defined herein as having a common center or axis. The concentric catheter device 100 includes a needle 102, a first catheter 104, and a second catheter 106. As shown in FIG. 2, the needle 102 has a needle proximal end 208 and a needle distal end 210. The needle proximal end 208 and the needle distal end 210 are longitudinally spaced apart by an elongate needle body 212. The term “longitudinal” is used herein to indicate a substantially vertical direction, in the orientation of FIG. 1. The needle 102 has a needle outer wall 214. The needle distal end 210 has a needle tip 216. The needle 102 can be, but is not limited to, a 22G needle. The needle 102 may have a needle inner lumen (not shown). The needle inner lumen may extend between the needle proximal end 208 and the needle tip 216. The needle inner lumen may be capable of having a guidewire (not shown) directed therethrough.

As shown in FIG. 3, the first catheter 104 has a first catheter proximal end 318 and a first catheter distal end 320. The first catheter proximal end 318 and the first catheter distal end 320 are longitudinally spaced apart by an elongate first catheter body 322. At least a portion of the first catheter distal end 320 may be inwardly tapered, as shown by first catheter tapered portion 321. The term “taper” is defined herein as a gradual reduction of thickness, diameter, or width in an elongated object, as is shown by the gradual diminution in diameter of the first catheter distal end 320 in FIG. 3. Further, the inward taper, such as the taper of the first catheter distal end 320, might include no expansion in diameter (or outward taper) distal to the first catheter body 322. The first catheter 104 has a first catheter outer wall 324. The first catheter 104 has a first catheter inner lumen 326. The first catheter distal end 320 has a first catheter open tip 328. The first catheter inner lumen 326 extends between the first catheter proximal end 318 and the first catheter open tip 328. The first catheter inner lumen 326 is larger in diameter than the needle outer wall 214. The first catheter proximal end 318 is capable of being attached to a fluid source (not shown). The first catheter 104 can be, but is not limited to, a 22G, an 18G, or a 16G catheter.

As shown in FIG. 4, the second catheter 106 has a second catheter proximal end 430 and a second catheter distal end 432. The second catheter proximal end 430 and the second catheter distal end 432 are longitudinally spaced apart by an elongate second catheter body 434. At least a portion of the second catheter distal end 432 may be inwardly tapered, as shown by second catheter tapered portion 433. The second catheter 106 has a second catheter outer wall 436. The second catheter 106 has a second catheter inner lumen 438. The second catheter distal end 432 has a second catheter open tip 440. The second catheter inner lumen 438 extends between the second catheter proximal end 430 and the second catheter open tip 440. The second catheter inner lumen 438 is larger in diameter than the first catheter outer wall 324. The second catheter proximal end 430 is capable of being attached to a fluid source (not shown). The second catheter 106 can be, but is not limited to, an 18G, a 16G, or a 14G catheter.

When the second catheter 106 is joined to the first catheter 104, the first catheter open tip 328 and at least a portion of the first catheter distal end 320 longitudinally extend distally from the second catheter open tip 440. That is, when joined together, the first catheter open tip 328 and at least a portion of the first catheter distal end 320 are not positioned within the second catheter inner lumen 438. Further, when the first catheter 104 is joined to the second catheter 106, the first catheter outer wall 324 is directly, or, alternatively, nondirectly, laterally adjacent to the second catheter inner lumen 438. That is, when the first catheter 104 is joined to the second catheter 106, the second catheter inner lumen 438 surrounds at least a portion of the first catheter outer surface 324. The term “lateral” is used herein to indicate a direction substantially perpendicular to the “longitudinal” direction, and is shown as the horizontal direction in the orientation of FIGS. 1-4.

When the needle 102 is joined to the first catheter 104, the needle tip 216 and at least a portion of the needle distal end 210 longitudinally extend distally from the first catheter open tip 328. That is, when joined together, the needle tip 216 and at least a portion of the needle distal end 210 are not positioned within the first catheter inner lumen 326. Further, when the needle 102 is joined to the first catheter 104, the needle outer wall 214 is directly, or, alternatively, nondirectly, laterally adjacent to the first catheter inner lumen 326. That is, when the needle 102 is joined to the first catheter 104, the first catheter inner lumen 326 surrounds at least a portion of the needle outer wall 214.

Additionally, when the needle 102 is joined to the first catheter 104, the needle outer wall 214 is directly, or, alternatively, nondirectly, laterally adjacent to the first catheter inner lumen 326, and when the combined needle 102 and first catheter 104 are joined to the second catheter 106, the needle outer wall 214 is directly, or, alternatively, nondirectly, laterally adjacent to the first catheter inner lumen 326 and the first catheter outer wall 324 is directly, or, alternatively, nondirectly, laterally adjacent to the second catheter inner lumen 438. That is, when the needle 102, the first catheter 104, and second catheter 106 are joined together, the first catheter inner lumen 326 laterally surrounds at least a portion of the needle outer wall 214 and the second catheter inner lumen 438 laterally surrounds at least a portion of the first catheter outer wall 324.

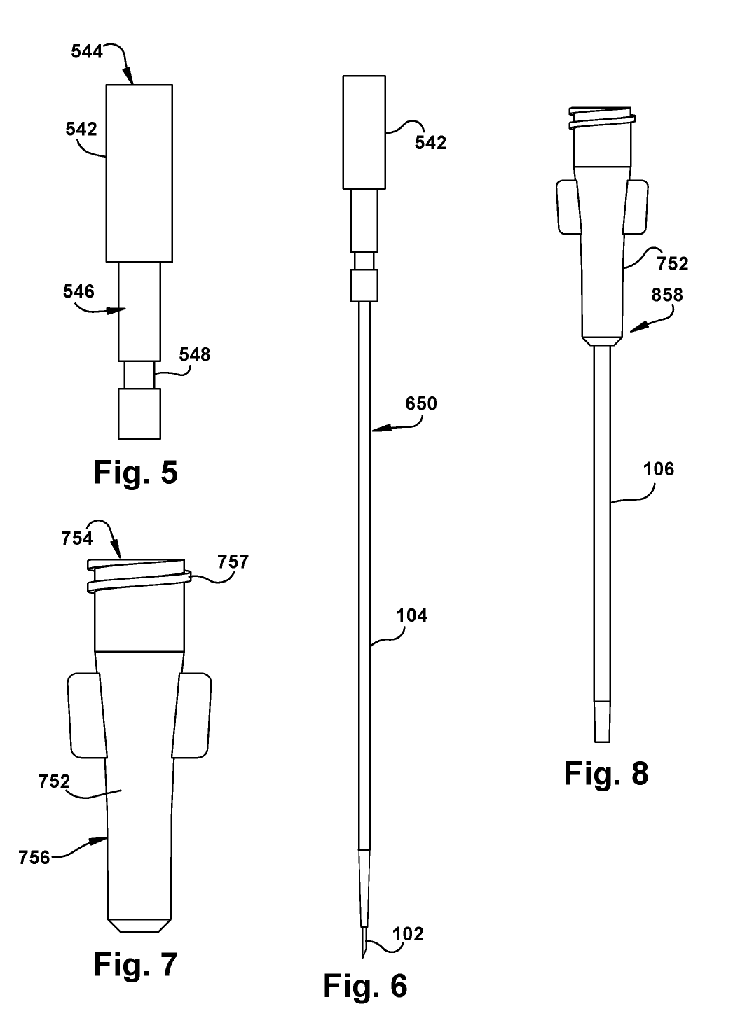

As shown in FIG. 5, the concentric catheter device 100 can include a first connector 542. The first connector 542 has a first connector inner lumen 544 and a first connector outer wall 546. The first connector inner lumen 544 is larger in diameter than the first catheter outer wall 324. The first connector outer wall 546 has a first connector locking mechanism 548. The first connector 542 can selectively accept at least one of the first catheter 104 and the needle 102. As shown in FIG. 6, at least a portion of the first catheter body 332 and at least a portion of the needle body 212 can be attached to the first connector inner lumen 544 in order to lock the needle 102 concentrically within the first catheter 104 to form an integral needle-first catheter-first connector unit 650. That is, when the needle 102, the first catheter 104, and the first connector 542 are joined together, the needle 102, the first catheter 104, and the first connector 542 can each have the same center longitudinal axis. The needle 102 and/or first catheter 104 can be attached to the first connector inner lumen 544 through the use of at least one of a snap-fit engagement, an adhesive engagement, a press-fit frictional engagement, any other suitable engagement, or any combination thereof. The first connector 542 is capable of being attached to a fluid source (not shown).

As shown in FIG. 7, the concentric catheter device 100 can include a second connector 752. The second connector 752 has a second connector inner lumen 754 and a second connector outer wall 756. At least a portion of the second connector inner lumen 754 is larger in diameter than the second catheter outer wall 436. At least a portion of the second connector inner lumen 754 is larger in diameter than at least a portion of the first connector 542. The second connector inner lumen 754 has a second connector locking mechanism (not shown). The second connector 752 can selectively accept at least one of the second catheter 106 and the first connector 542. The second connector outer wall 756 can have a second connector outer wall locking mechanism 757.

As shown in FIG. 8, at least a portion of the second catheter body 434 can be attached to the second connector inner lumen 754 to form an integral concentric second catheter-second connector unit 858. That is, when the second catheter 106 and the second connector 752 are joined together, the second catheter 106 and the second connector 752 can each have the same center longitudinal axis. The second catheter 106 can be attached to the second connector inner lumen 754 through the use of at least one of a snap-fit engagement, an adhesive engagement, a press-fit frictional engagement, any other suitable engagement, or any combination thereof. The second connector locking mechanism can be attached to the first connector locking mechanism 548 through the use of at least one of a snap-fit engagement, an adhesive engagement, a press-fit frictional engagement, any other suitable engagement, or any combination thereof. The second connector 752 is capable of being attached to a fluid source (not shown).

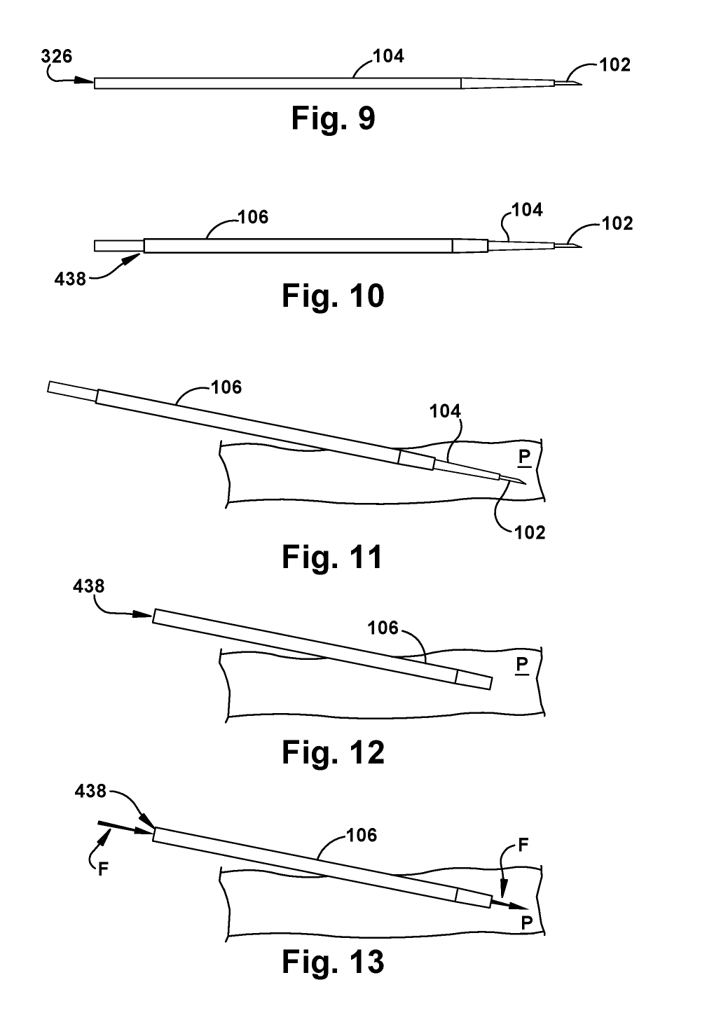

In use, a concentric catheter device 100, as described above, is provided to the user. As shown in FIG. 9, the user can insert the needle 102 into the first catheter inner lumen 326, with the needle 102 and the first catheter 104 being concentrically arranged relative to one another. That is, when the needle 102 is joined to the first catheter 104, the first catheter 104 and the inserted needle 102 can each have the same center longitudinal axis. As shown in FIG. 10, the user can insert the first catheter 104, with the inserted needle 102, into the second catheter inner lumen 438, wherein the first catheter 104, the needle 102, and the second catheter 106 are concentrically arranged relative to one another. That is, when the needle 102, the first catheter 104, and the second catheter 106 are joined together, the second catheter 106, the inserted first catheter 104, and the inserted needle 102 can each have the same center longitudinal axis.

As shown in FIG. 11, the first catheter 104, the second catheter 106, and the needle 102 can be collectively inserted into a patient vessel lumen P. As shown in FIG. 12, the needle 102 and the first catheter 104 can be removed from the second catheter inner lumen 438, while maintaining the second catheter 106 in the patient vessel lumen P. The user can attach a fluid delivery source (not shown) to the second catheter 106. As shown in FIG. 13, the user can selectively direct fluid F to the patient vessel lumen P, wherein fluid F from the fluid delivery source is directed through the second catheter inner lumen 438 to the patient vessel lumen P. For example, the user can use a thumb lever, foot pedal, or any other user-actuable device to activate the flow of fluid from the fluid source to the patient vessel lumen P.

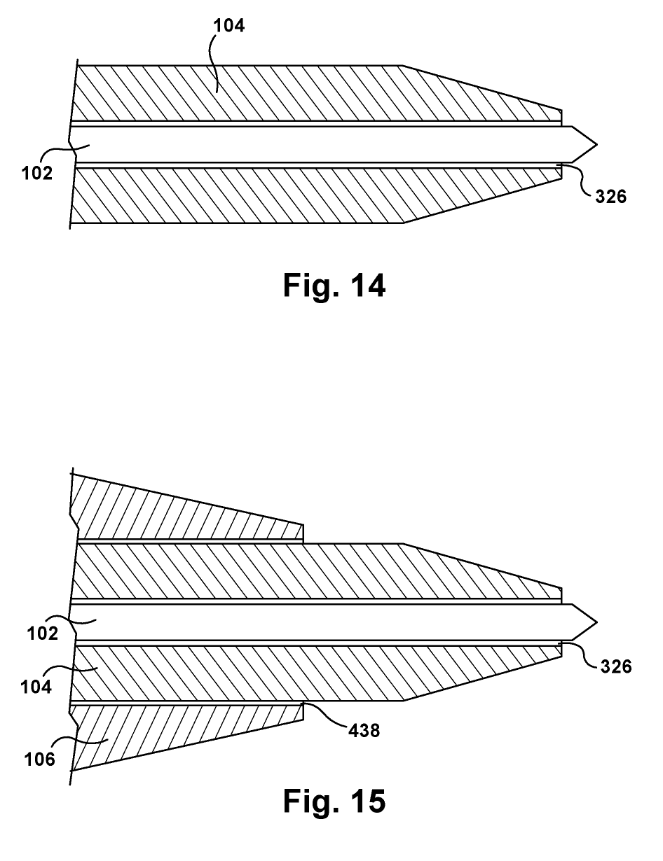

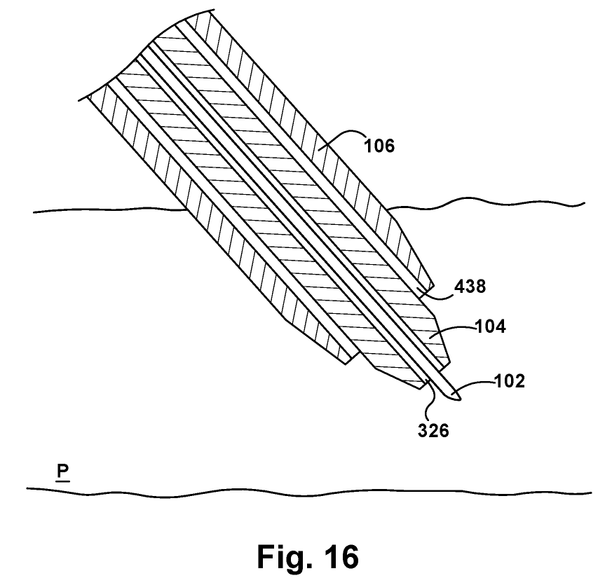

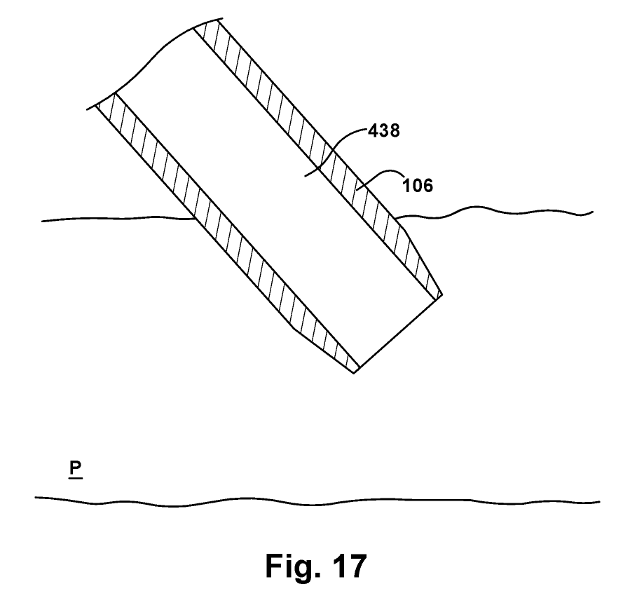

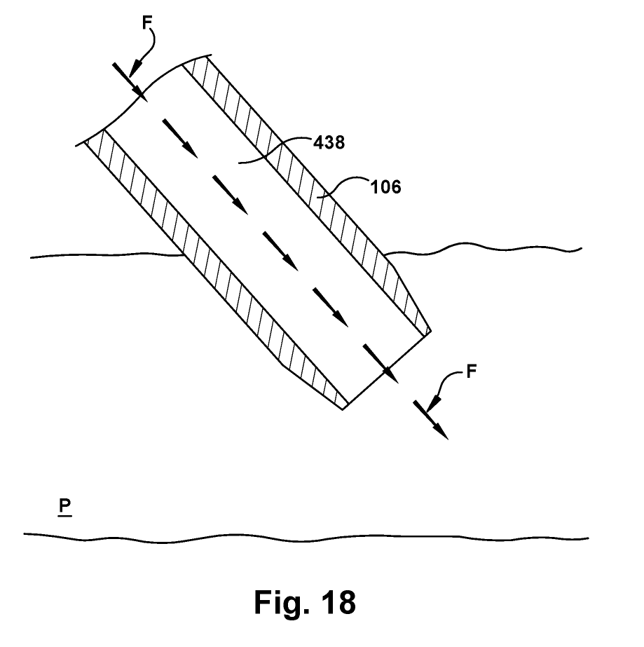

FIGS. 14-18 depict a similar sequence to the sequence depicted in FIGS. 9-13, with a cross-sectional view of the concentric catheter device 100. Aspects of the sequence of operation of FIGS. 9-13 described above will not be repeated for the sequence depicted in FIGS. 14-18, for brevity.

Instead of removing both the needle 102 and first catheter 104 from the second catheter inner lumen 438 after the concentric catheter device 100 has been inserted into the patient vessel lumen P, the needle 102 can be removed from the first catheter inner lumen 326, while maintaining the first catheter 104 in place within the second catheter 106 and the patient vessel lumen P. For example, the user can insert the needle 102 into the first catheter inner lumen 326, with the needle 102 and the first catheter 104 being concentrically arranged relative to one another. The user can insert the first catheter 104, with the inserted needle 102, into the second catheter inner lumen 438, wherein the first catheter 104, the needle 102, and the second catheter 106 are concentrically arranged relative to one another. This sequence is shown schematically in the cross-sectional views of FIGS. 14-18.

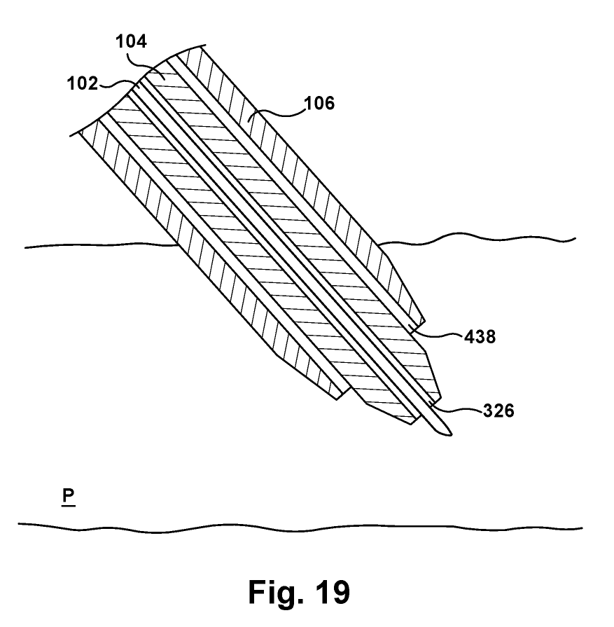

FIGS. 19-21 depict a similar sequence to the sequence depicted in FIGS. 9-13, with a cross-sectional view of the concentric catheter device 100. Aspects of the sequence of operation of FIGS. 9-13 described above will not be repeated for the sequence depicted in FIGS. 19-21, for brevity.

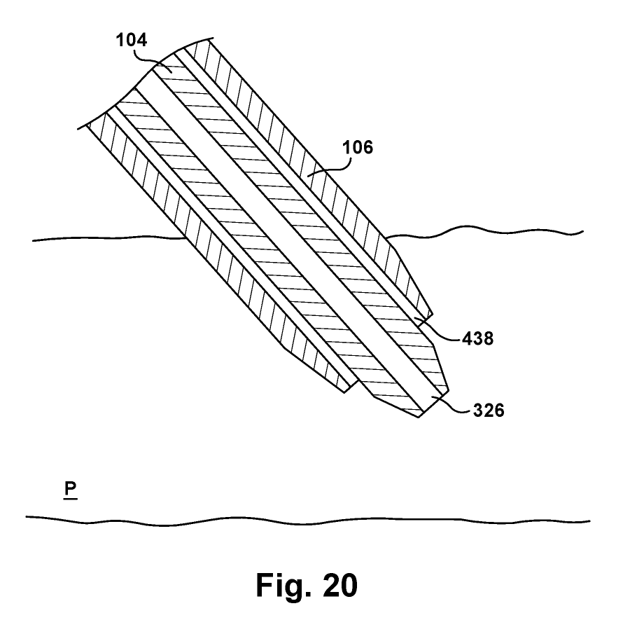

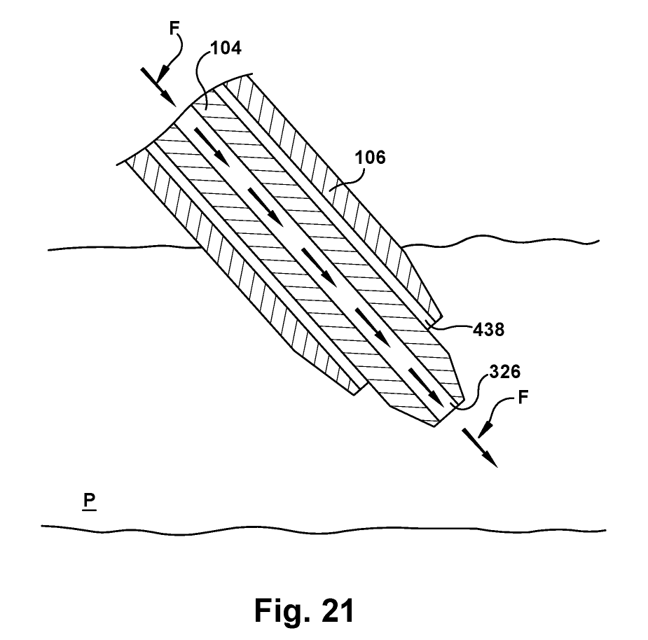

As shown in FIG. 19, the first catheter 104, the second catheter 106, and the needle 102 can be collectively inserted into a patient vessel lumen P. As shown in FIG. 20, the user can remove the needle 102 from the first catheter inner lumen 326, while maintaining the first catheter 104 in place within the second catheter 106 and the patient vessel lumen P. A fluid delivery source can be attached to the first catheter 104. As shown in FIG. 21, the user can selectively direct fluid F to the patient vessel P, wherein fluid F from the fluid delivery source is directed through the first catheter inner lumen 326 to the patient vessel lumen P. For example, the user can use a thumb lever, foot pedal, or any other user-actuable device to activate the flow of fluid from the fluid source to the patient vessel lumen P.

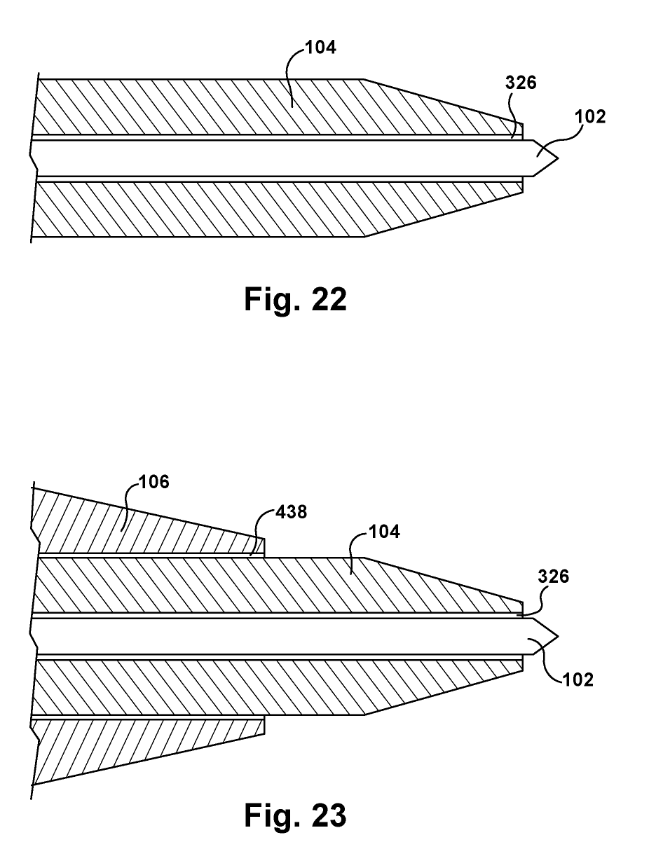

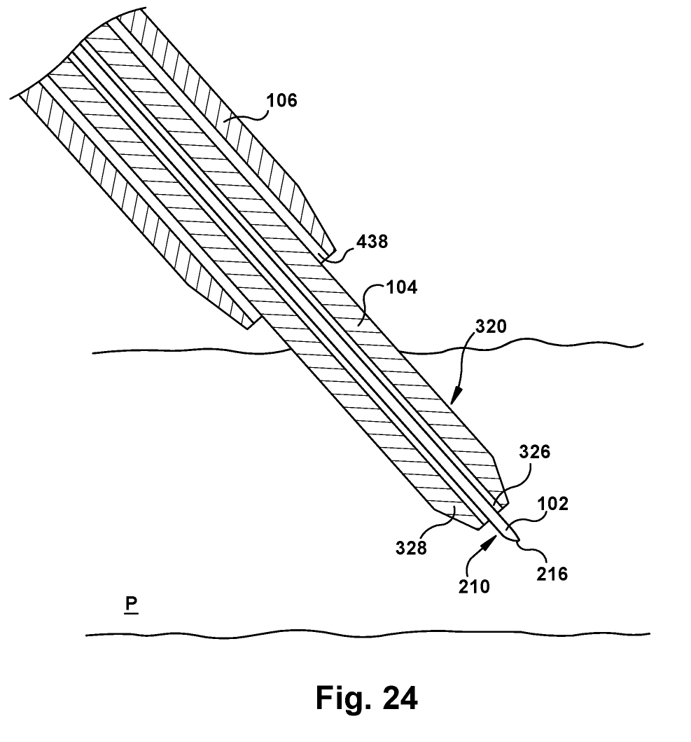

Alternatively, in use, a concentric catheter device 100, as described above, is provided to the user. As shown in FIG. 22, the user can insert the needle 102 into the first catheter inner lumen 326, with the needle 102 and the first catheter 104 being concentrically arranged relative to one another. As shown in FIG. 23, the user can insert the first catheter 104, with the inserted needle 102, into the second catheter inner lumen 438, wherein the first catheter 104, the needle 102, and the second catheter 106 are concentrically arranged relative to one another. As shown in FIG. 24, the needle tip 216, at least a portion of the needle distal end 210, the first catheter open tip 328, and at least a portion of the first catheter distal end 320 can be inserted into the patient vessel lumen P.

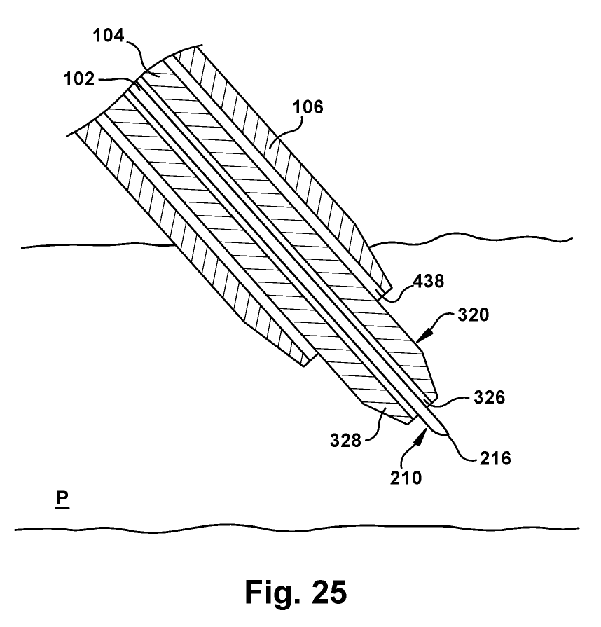

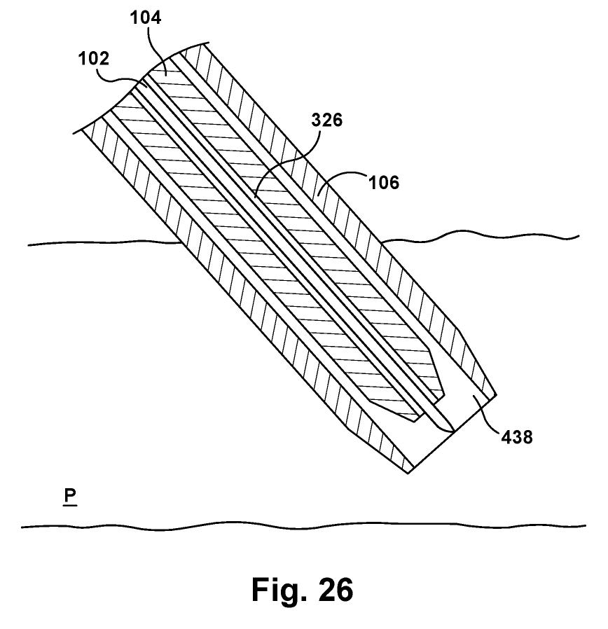

As shown in FIGS. 25-26, at least a portion of the second catheter 106 can be inserted into the patient vessel lumen P, with the second catheter 106 being slidably translated longitudinally along the first catheter 104 into the patient vessel lumen P, while the first catheter 104 is at least partially within the patient vessel lumen P. In a similar sequence as described above, the user can remove at least one of the needle 102 and first catheter 104 from the second catheter inner lumen 438, attach a fluid delivery source to the first catheter 104 or the second catheter 106, and selectively direct fluid F to the patient vessel lumen P. For example, the user can use a thumb lever, foot pedal, or any other user-actuable device to activate the flow of fluid from the fluid source to the patient vessel lumen P.

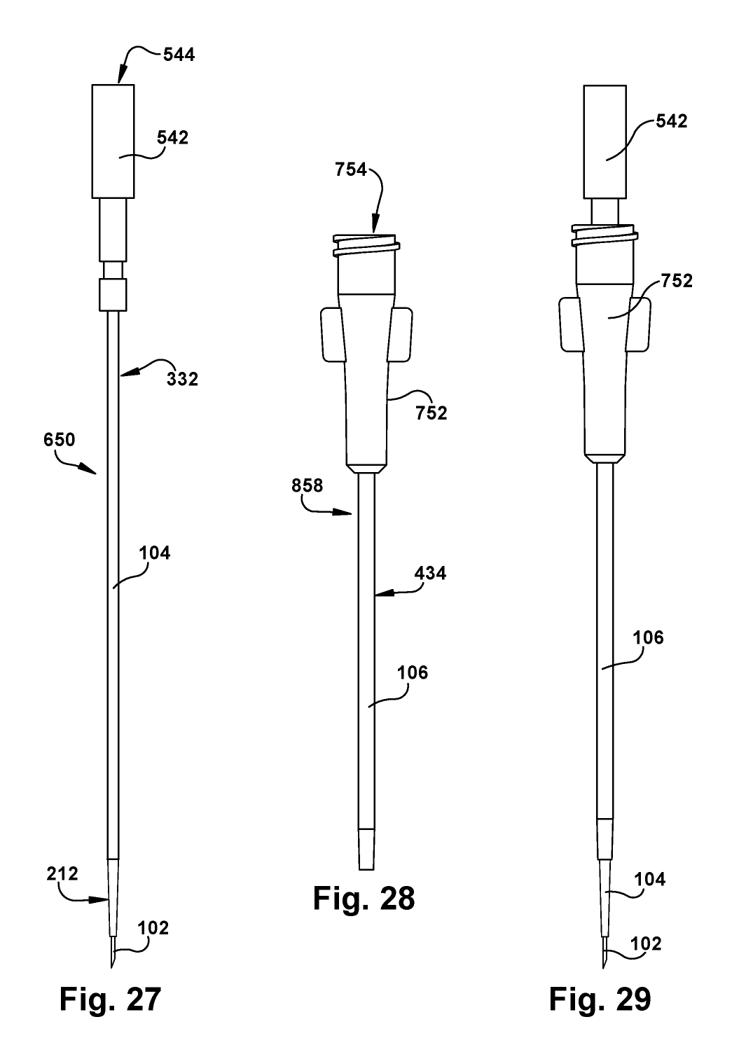

Alternatively, FIGS. 27-32 depict a similar sequence to the sequence depicted in FIGS. 9-13, with a provided first connector 542 and second connector 752. Aspects of the sequence of operation of FIGS. 9-13 described above will not be repeated for the sequence depicted in FIGS. 27-32, for brevity. The user can insert the needle 102 into the first catheter inner lumen 326, with the needle 102 and the first catheter 104 being concentrically arranged relative to one another. As shown in FIG. 27, the user can attach at least one of the first catheter body 332 and the needle body 212 to the first connector inner lumen 544 in order to lock the needle 102 within the first catheter 104 to form a concentrically integral needle-first catheter-first connector unit 650. That is, when the needle 102, the first catheter 104, and the first connector 542 are joined together, the needle 102, the first catheter 104, and the first connector 542 can each have the same center longitudinal axis.

As shown in FIG. 28, the user can attach at least a portion of the second catheter body 434 to the second connector inner lumen 754 to form an integral concentric second catheter-second connector unit 858. That is, when the second catheter 106 and the second connector 752 are joined together, the second catheter 106 and the second connector 752 can each have the same center longitudinal axis. As shown in FIG. 29, the second connector 752, which has second catheter 106 attached therein, can be attached to the first connector 542, which has at least one of the first catheter 104 and needle 102 attached therein. That is, the first connector 542, which has at least one of the first catheter 104 and needle 102 attached therein, can be inserted into the second connector 752, which has the second catheter 106 attached therein, until the first connector locking mechanism 548 engages the second connector locking mechanism (not shown).

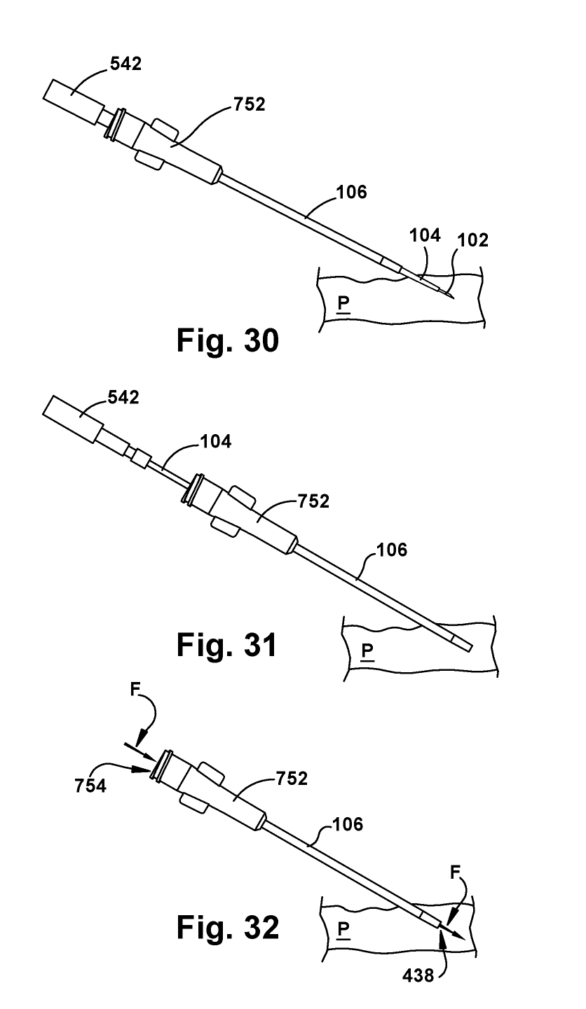

As shown in FIG. 30, the needle 102, the first catheter 104, and the second catheter 106, which are concentrically arranged relative to one another and held together by the attached first connector 542 and second connector 752, can be collectively inserted into the patient vessel lumen P. As shown in FIG. 31, the first connector 542, and the attached needle 102 and first catheter 104, can be removed from the second connector 752, and attached second catheter 106. A fluid delivery source can be attached to the second connector 752. As shown in FIG. 32, the user can selectively direct fluid F to the patient vessel lumen P, wherein fluid F from the fluid delivery source is directed through the second connector inner lumen 754, through the second catheter inner lumen 438, and out to the patient vessel lumen P. For example, the user can use a thumb lever, foot pedal, or any other user-actuable device to activate the flow of fluid from the fluid source to the patient vessel lumen P.

Alternatively, instead of removing the needle-first catheter-first connector unit 650, only the needle 102 is removed from the first connector 542, with attached first catheter 104, and the second connector 752, with attached second catheter 106. A fluid delivery source can be attached to the first connector 542. The user can selectively direct fluid F to the patient vessel lumen P, wherein fluid F from the fluid delivery source is directed through the first connector inner lumen 544, through the first catheter inner lumen 326, and out to the patient vessel lumen P. For example, the user can use a thumb lever, foot pedal, or any other user-actuable device to activate the flow of fluid from the fluid source to the patient vessel lumen P.

Alternatively, similarly to the sequence depicted in FIGS. 22-26, after the needle-first catheter-first connector unit 650 is joined to the second catheter-second connector unit 858, the needle tip 216, at least a portion of the needle distal end 210, the first catheter open tip 328, and at least a portion of the first catheter distal end 320 can be inserted into the patient vessel lumen P. At least a portion of the second catheter 106 can be inserted into the patient vessel lumen P, with the second catheter-second connector unit 858 being disengaged from the needle-first catheter-first connector unit 650 and being slidably translated longitudinally along the first catheter 104 into the patient vessel lumen P, while the needle-first catheter-first connector unit 650 at least partially within the patient vessel lumen P. In a similar sequence as described above, the user can remove at least one of the needle 102 and first catheter 104 from the second catheter inner lumen 438, attach a fluid delivery source to the first connector 542 or the second connector 752, and selectively direct fluid F to the patient vessel lumen P. For example, the user can use a thumb lever, foot pedal, or any other user-actuable device to activate the flow of fluid from the fluid source to the patient vessel lumen P.

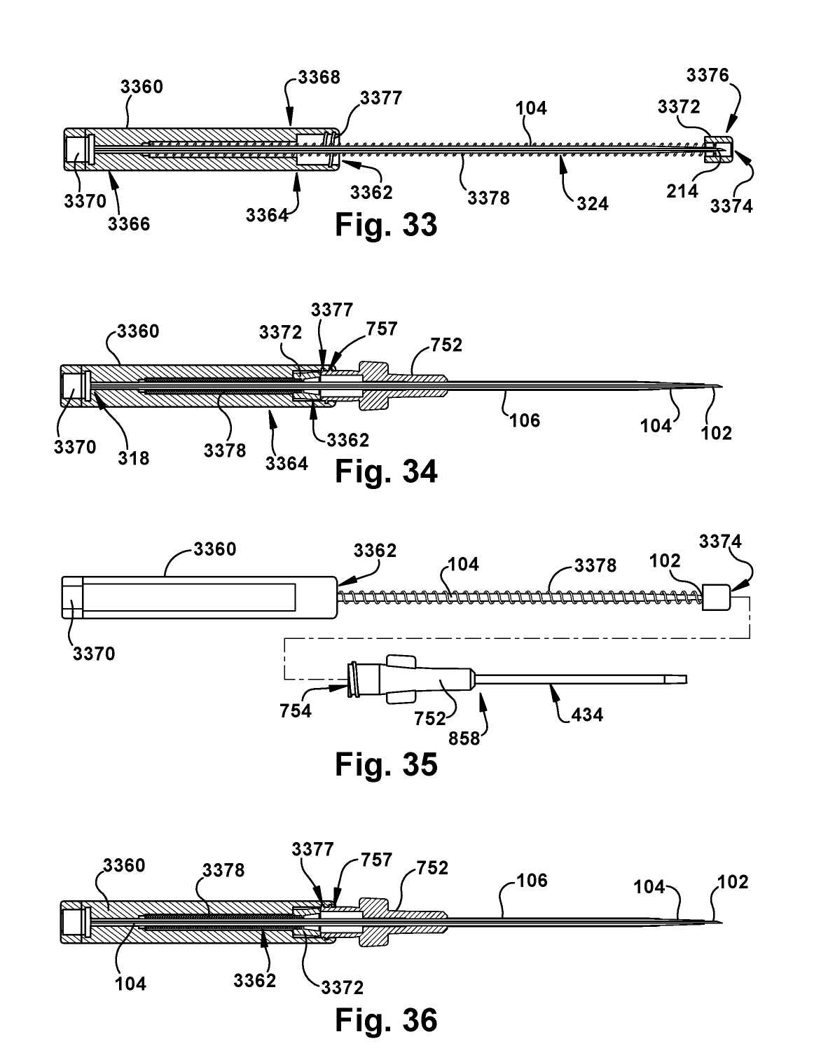

FIGS. 33-34 depict a surrounding mechanism 3360 that may be used with any of the configurations discussed above. The surrounding mechanism 3360 includes a surrounding mechanism inner lumen 3362 and a surrounding mechanism outer wall 3364. The surrounding mechanism outer wall 3364 could be at least partially made from a transparent and/or translucent material, as shown in the Figures, to allow the user to view the surrounding mechanism inner lumen 3362. At least a portion of the surrounding mechanism inner lumen 3362 is larger in diameter than at least one of the first connector outer wall 546 and second connector outer wall 756. The surrounding mechanism 3360 has a surrounding mechanism proximal end 3366 and a surrounding mechanism distal end 3368. The surrounding mechanism proximal end 3366 can include a transparent and/or translucent window 3370. The window 3370 allows the user to view at least a portion of the surrounding mechanism inner lumen 3362 through the surrounding mechanism outer wall 3364.

The surrounding mechanism 3360 includes a needle cover 3372. The needle cover 3372 has a needle cover inner lumen 3374 and needle cover outer surface 3376. The needle cover inner lumen 3374 is larger in diameter than at least one of the first catheter outer wall 324 and needle outer wall 214. The needle cover outer wall 3376 is smaller in diameter than at least a portion of the surrounding mechanism inner lumen 3362. The surrounding mechanism 3360 has a biasing member 3378, such as, but not limited to, a spring. The biasing member 3378 is attached to both a portion of the surrounding mechanism inner lumen 3362 and a portion of the needle cover 3372 (such as, but not limited to, the needle cover inner lumen 3374).

The surrounding mechanism inner lumen 3362 has a surrounding mechanism locking member 3377. As shown in FIG. 34, the surrounding mechanism locking member 3377 is capable of engaging the second connector outer wall locking mechanism 757. The second connector outer wall locking member 757 can be attached to the surrounding mechanism locking mechanism through the use of at least one of a snap-fit engagement, an adhesive engagement, a press-fit frictional engagement, any other suitable engagement, or any combination thereof.

In use, the concentric catheter device 100 is provided to the user. The user can insert the needle 102 into the first catheter inner lumen 326, with the needle 102 and the first catheter 104 being concentrically arranged relative to one another. As shown in FIG. 35, the user can insert the first catheter 104, with inserted needle 102, through the needle cover inner lumen 3374, through the biasing member 3378, and into the surrounding mechanism inner lumen 3362 until at least a portion of at least one of first catheter proximal end 318 and the needle proximal end 208 are located within at least a portion of the surrounding mechanism inner lumen 3362 that is viewable by the user through the window 3370. The user can attach at least a portion of the second catheter body 434 to the second connector inner lumen 754 to form an integral concentric second catheter-second connector unit 858.

As shown in FIG. 36, the second connector 752, which has second catheter 106 attached therein, can be attached to the surrounding mechanism 3360, which has at least one of the first catheter 104 and needle 102 inserted therein. That is, the second connector 752, which has the second catheter 106 attached therein, can be inserted into the surrounding mechanism 3360, which has at least one of the first catheter 104 and needle 102 inserted therein, until the surrounding mechanism locking member 3377 engages the second connector outer wall locking mechanism 757. As the surrounding mechanism 3360, with inserted first catheter 104 and needle 102, is inserted into the second connector 752, the needle cover 3372 is longitudinally translated into the surrounding mechanism inner lumen 3362. The translation of the needle cover 3372 into the surrounding mechanism inner lumen 3362 causes the biasing member 3378 to be compressed within the surrounding mechanism inner lumen 3362. When the surrounding mechanism 3360 is engaged to the second connector 752, the needle cover 3372 abuts the second connector 752, which holds the biasing member 3378 in a compressed state.

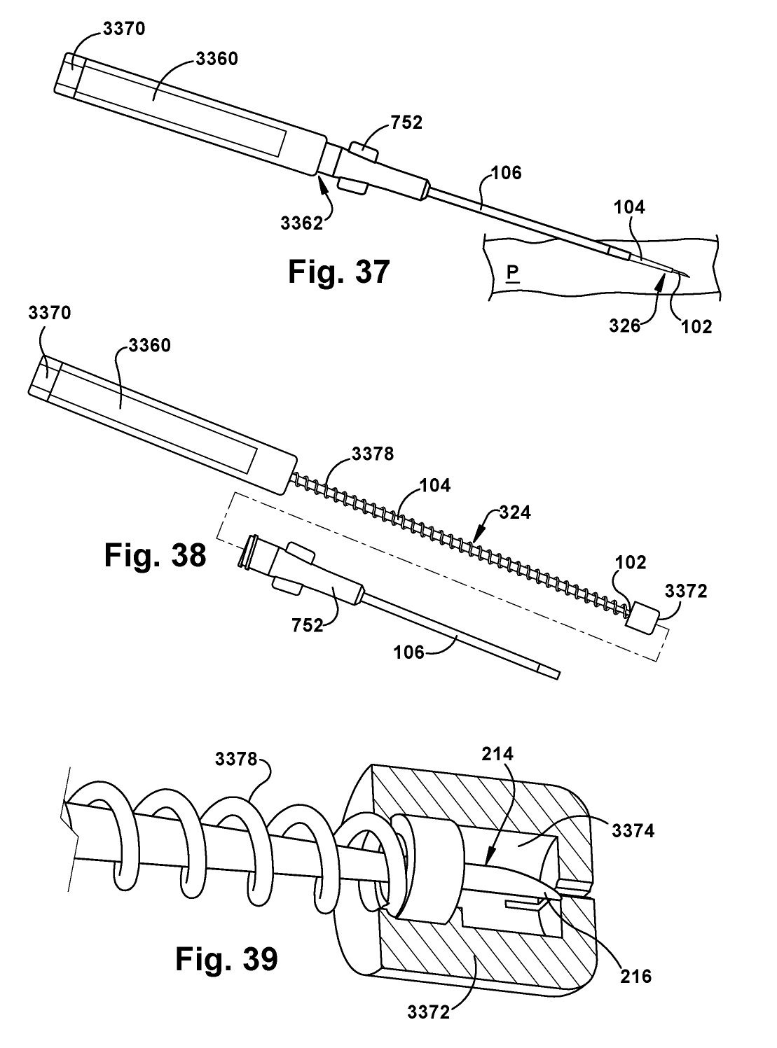

As shown in FIG. 37, the needle 102, the first catheter 104, and the second catheter 106, which are concentrically arranged relative to one another and held together by the attached surrounding mechanism 3360 and second connector 752, can be collectively inserted into the patient vessel lumen P. Once the concentric catheter device 100 is inserted into the patient vessel lumen P, blood from the patient vessel lumen P may flow through the first catheter inner lumen 326 and into the portion of the surrounding mechanism inner lumen 3362 that is viewable to the user through the window 3370. Once the user detects blood with the surrounding mechanism inner lumen 3362 by a visual confirmation through the window 3370, the user is aware that the concentric catheter device 100 has been inserted into the patient vessel lumen P.

The surrounding mechanism 3360, and the attached needle 102 and first catheter 104, can be removed from the second connector 752, and attached second catheter 106. As the surrounding mechanism 3360, and inserted needle 102 and first catheter 104, are removed from the second connector 752 and attached second catheter 106, the biasing member 3378 decompresses, which causes the biasing member 3378 to increasingly be directly, or, alternatively, nondirectly, laterally adjacent to the first catheter outer wall 324. That is, as the surrounding mechanism 3360, and inserted needle 102 and first catheter 104, are removed from the second connector 752, and attached second catheter 106, the biasing member 3378 increasingly surrounds at least a portion of the first catheter outer wall 324 that was not surrounded by the biasing member 3378 when the biasing member 3378 was compressed.

As shown in FIG. 38, when the surrounding mechanism 3360, and inserted needle 102 and first catheter 104, are removed from the second connector 752, and attached second catheter 106, the needle cover 3372 no longer abuts the second connector 752, which causes the biasing member 3378 to be fully decompressed. When the biasing member 3378 is fully decompressed, the biasing member 3378 causes the biasing member 3378 to be directly, or, alternatively, nondirectly, laterally adjacent to the first catheter outer wall 324 and the needle outer wall 214. That is, when the biasing member 3378 is fully decompressed, the biasing member 3378 surrounds at least a portion of the first catheter outer wall 324 and at least a portion of the needle outer wall 214 that was not surrounded by the biasing member 3378 when the biasing member 3378 was compressed. Further, as shown in FIG. 39, when the biasing member 3378 is fully decompressed, the needle cover inner lumen 3374 is directly, or, alternatively, nondirectly, laterally adjacent to the needle outer wall 214 and needle tip 216. That is, when the biasing member 3378 is fully decompressed, the needle cover 3372 surrounds the needle tip 216 and at least a portion of the needle outer wall 214.

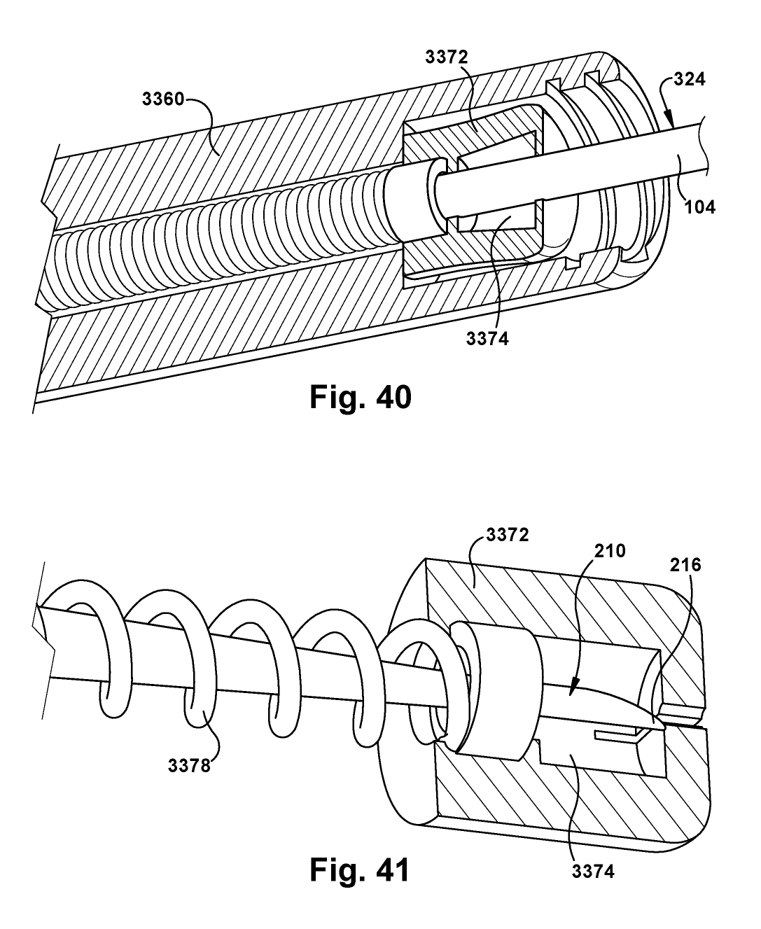

As shown in FIG. 40, the needle cover 3372 may be made from an elastic material. In this depicted configuration, the needle cover inner lumen 3374 may include a portion that is smaller in diameter than the first catheter outer wall 324. When the first catheter 104, with inserted needle 102, is inserted into the needle cover 3372, the elastic nature of the needle cover 3372 allows the portion of the needle cover inner lumen 3374 that is smaller in diameter than the first catheter outer wall 324 to expand in order to receive the first catheter 104, with inserted needle 102. Further, as shown in FIG. 41, when biasing member 3378 is fully decompressed, as described above, the elastic nature of the needle cover 3372 causes the needle cover 3372 to return to its original position, which results in the needle tip 216 and at least a portion of the needle distal end 210 being surrounded by the needle cover inner lumen 3374.

The needle 102, the first catheter 104, the second catheter 106, the first connector 542, the second connector 752, and/or surrounding mechanism 3060 can each be at least partially formed from silicone, polyethylene, polypropylene, stainless steel, titanium, any other biocompatible material, or any combination thereof.

It is contemplated that the concentric catheter device 100 may provide the user with the ability to insert a large bore catheter, such as the second catheter 106, into a patient vessel lumen P that may have been constricted, possibly due to hypotension. In the case of a constricted patient vessel lumen P, it may be more desirable to insert a small bore catheter, such as the first catheter 104, in order to reduce potential trauma, complications, and/or risks to the patient that may result from introducing a large bore catheter directly into a constricted patient vessel lumen. However, in certain circumstances, it may be more desirable to use a large bore catheter, such as the second catheter 106, to introduce blood and/or fluid into a constricted patient vessel lumen P at a faster flow rate than what a small bore catheter would allow.

The concentric catheter device 100 assists the user in introducing a large bore catheter into a constricted patient vessel lumen P due to the gradually increasing diameter of the combinations of catheters provided in the concentric catheter device 100. For example, the user can insert the needle 102, such as a 22G needle, into the first catheter 104, such as a 20G catheter. Then, the user can insert the first catheter 104 (including the tapered portion 321), with the attached needle 102 therein, into the second catheter 106, such as a 16G catheter. In this combined arrangement, at least a portion of the needle distal end 210 longitudinally extends distally from the first catheter open tip 328, and at least a portion of the first catheter distal end 320 longitudinally extends distally from the second catheter open tip 440 to form a concentric catheter device 100 that gradually increases in diameter. Additionally, in the combined arrangement, at least a portion of the first catheter tapered portion 321 may also extend distally from the second catheter open tip 440. Thus, as the concentric catheter device 100 is introduced to a constricted patient vessel lumen P, first, the needle 102 is introduced, then, the first catheter 104 is introduced, and then, the second catheter is introduced.

Therefore, a constricted patient vessel lumen P, in which it is desirable to insert only a small bore catheter, such as the first catheter 104, into, is gradually stretched using first catheter tapered portion 321 to accept a large bore catheter, such as the second catheter 106. This gradual increase in the diameter of the concentric catheter device 100 allows a large bore catheter to be introduced into a constricted patient vessel lumen P, which can typically only accept a small bore catheter, while reducing the potential trauma, complications, and/or risks to the patient that results from introducing a large bore catheter into a constricted patient vessel lumen P.

Additionally, at least a portion of the second catheter distal end 432 can have an inward taper in the range of about six (6) degrees and at least a portion of the first catheter distal end 320 can have an inward taper in the range of about four (4) degrees, in order to form concentric catheter device 100 with a gradual increase in diameter. However, any suitable degree of taper may be used.

It is contemplated that the concentric catheter device 100 can be used to remove blood and/or fluid from a patient vessel lumen P using a similar sequence of operation as described above. For example, the concentric catheter device 100 can be inserted into a patient vessel lumen P. At least one of the needle 102 and first catheter 104 can be removed from the second catheter 106. A vacuum force, instead of, or in addition to, a fluid source, can be applied one of the second catheter 106 or first catheter 104 to extract blood and/or fluid from the patient vessel lumen.

It is contemplated that the concentric catheter device 100 can be operated using a combination of the needle 102 and first catheter 104. In this configuration, the user can introduce the first catheter to a patient vessel lumen. The user can then direct fluid to the patient vessel lumen in a similar manner as described above. Additionally, if a user desires to use a large bore catheter (such as a catheter larger in diameter than the first catheter outer surface 324), the user can join the first catheter 104 to the needle 102 and place the first catheter 104, with inserted needle 102, within a commercially available large bore catheter.

It is also contemplated that the second catheter 106 can be combined, in a similar sequence as described above, with at least one of a commercially available needle and a commercially available small bore catheter (such as a catheter smaller in diameter than the second catheter inner lumen 438) to create a concentric catheter device 100. In this configuration, a similar sequence of operation as described above may be used.

It is contemplated that the first catheter body 322 may be longitudinally longer than the second catheter body 434. For example, the first catheter body 322 can be, but is not limited to, two (2) to three (3) times as long as the second catheter body 434. In this configuration, the first catheter 104 may be introduced to the patient vessel lumen P prior to the introduction of the second catheter 106. In use, once the first catheter 104 is introduced into the patient vessel lumen P, the user can direct fluids F, such as, but not limited to, warm fluids, to the patient vessel lumen P in order to dilate the patient vessel lumen P. Once the patient vessel lumen P is dilated a desired amount, the user can introduce the second catheter 106 to the patient vessel lumen P, as described above. Additionally, at least a portion of the second catheter distal end 432 can have an inward taper of four (4) degrees and at least a portion of the first catheter distal end 320 can have an inward taper of four (4) degrees in order to form concentric catheter device 100 with a gradual increase in diameter. However, any suitable degree of taper may be used.

Although the concentric catheter device 100 has been described to be used in a procedure that may involve a patient vessel lumen P, it is to be understood that the concentric catheter device 100 may be used in any similar procedure that may involve the introduction/extraction of fluids to/from a target site, in which a large diameter device is desired, but, due to size related issues, only a small diameter device can be used.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many applications. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages can be obtained from a study of the drawings, the disclosure, and the appended claims.

1-26. (canceled)

27. A system for selectively accessing an interior lumen of a patient vessel, comprising:
a needle, the needle having an elongate needle body and a needle distal end;
a first catheter, the first catheter having an elongate first catheter body and a first catheter distal end, the first catheter body defining a first catheter inner lumen, at least a portion of the first catheter inner lumen being larger in diameter than the needle body and the needle distal end, the first catheter distal end having a first catheter open tip through which the needle distal end at least partially extends; and
a second catheter, the second catheter having an elongate second catheter body and a second catheter distal end, the second catheter body defining a second catheter inner lumen, the second catheter inner lumen being larger in diameter than the first catheter body and the first catheter distal end, the second catheter distal end having a second catheter open tip, the second catheter being capable of being attached to a fluid source;
wherein the needle and the first catheter are joined together to be concentrically arranged relative to one another; and
wherein the first catheter and the second catheter are slidingly joined together to be concentrically arranged relative to one another.

28. The system of claim 27, including a first connector, the first connector having a first connector inner lumen and a first connector outer surface, the first connector inner lumen being larger in diameter than at least one of the first catheter body, first catheter distal end, and needle body, the first connector outer surface having a first connector locking mechanism, the first connector selectively accepting at least one of the first catheter and the needle.

29. The system of claim 28, including a second connector, the second connector having a second connector inner lumen and a second connector outer surface, at least a portion of the second connector inner lumen being larger in diameter than the second catheter body and second catheter distal end, at least a portion of the second connector inner lumen being larger in diameter than at least a portion of the first connector, the second connector inner lumen having a second connector locking mechanism, the second connector selectively accepting at least one of the second catheter and the first connector.

30. The system of claim 27, wherein at least a portion of the first catheter distal end is inwardly tapered, and wherein at least a portion of the second catheter distal end is inwardly tapered.

31. The system of claim 27, wherein at least a portion of the needle body and the first catheter are fixedly joined together to be concentrically arranged to one another.

32. The system of claim 27, wherein the needle body and the first catheter are slidingly joined together to be concentrically arranged to one another.

33. A method for selectively accessing an interior lumen of a patient vessel, the method comprising:
providing a system for selectively accessing an interior lumen of a patient vessel including:
a needle, the needle having an elongate needle body and a needle distal end;
a first catheter, the first catheter having an elongate first catheter body and a first catheter distal end, the first catheter body defining a first catheter inner lumen, at least a portion of the first catheter inner lumen being larger in diameter than the needle body and the needle distal end, the first catheter distal end having a first catheter open tip through which the needle distal end at least partially extends; and
a second catheter, the second catheter having an elongate second catheter body and a second catheter distal end, the second catheter body defining a second catheter inner lumen, the second catheter inner lumen being larger in diameter than the first catheter body and the first catheter distal end, the second catheter distal end having a second catheter open tip, the second catheter being capable of being attached to a fluid source;
inserting the needle into the first catheter inner lumen, with the needle and the first catheter being concentrically arranged relative to one another;
inserting the first catheter, with the inserted needle, into the second catheter inner lumen, with the first catheter, the needle, and the second catheter being concentrically arranged relative to one another;
collectively inserting the first catheter, the second catheter, and the needle into a patient vessel lumen; and
removing at least one of the needle and the first catheter from the second catheter inner lumen, while maintaining the second catheter in the patient vessel lumen.

34. The method of claim 33, including:
providing a first connector, the first connector having a first connector inner lumen and a first connector outer surface, the first connector inner lumen being larger in diameter than at least one of the first catheter body, the first catheter distal end, and the needle body, the first connector outer surface having a first connector locking mechanism, the first connector selectively accepting at least one of the first catheter and the needle; and
attaching at least one of the at least a portion of the first catheter body and at least a portion of the needle body to the first connector inner lumen in order to lock the needle concentrically within the first catheter to form an integral needle-first catheter-first connector unit.

35. The method of claim 34, including:
providing a second connector, the second connector having a second connector inner lumen and a second connector outer surface, at least a portion of the second connector inner lumen being larger in diameter than the second catheter body and second catheter distal end, at least a portion of the second connector inner lumen being larger in diameter than at least a portion of the first connector, the second connector inner lumen having a second connector locking mechanism, the second connector selectively accepting at least one of the second catheter and the first connector; and
attaching at least a portion of the second catheter body to the second connector inner lumen to form an integral concentric second catheter-second connector unit.

36. The method of claim 35, including attaching the second connector, which has second catheter attached therein, to the first connector, which has at least one of the first catheter and needle attached therein, wherein the first connector, which has at least one of the first catheter and needle attached therein, is inserted into the second connector, which has the second catheter attached therein, until the first connector locking mechanism engages the second connector locking mechanism.

37. The method of claim 36, including:
collectively inserting the needle, the first catheter, and the second catheter, which are concentrically arranged relative to one another and held together by the attached first connector and second connector, into the patient vessel lumen;
removing the first connector, and the attached needle and first catheter, from the second connector, and attached second catheter;
attaching a fluid delivery source to the second connector; and
selectively directing fluid to the patient vessel lumen, wherein fluid from the fluid delivery source is directed through the second connector inner lumen, through the second catheter inner lumen, and out to the patient vessel lumen.

38. The method of claim 36, including:
collectively inserting the needle, the first catheter, and the second catheter, which are concentrically arranged relative to one another and held together by the attached first connector and second connector, into the patient vessel lumen;
removing the needle and the first catheter from the second catheter;
attaching a fluid delivery source to the second connector; and
selectively directing fluid to the patient vessel lumen, wherein fluid from the fluid delivery source is directed through the second connector inner lumen, through the second catheter inner lumen, and out to the patient vessel lumen.

39. The method of claim 33, including:
inserting at least a portion of the needle distal end and at least a portion of the first catheter distal end into the patient vessel lumen; and
inserting the second catheter into the patient vessel lumen, wherein the second catheter is slidably translated longitudinally along the first catheter into the patient vessel lumen with the first catheter at least partially within the patient vessel lumen.

40. The method of claim 33, including:
removing the needle from the first catheter inner lumen, while maintaining the first catheter in place within the second catheter and the patient vessel lumen;
attaching a fluid delivery source to the first catheter; and
selectively directing fluid to the patient vessel lumen, wherein fluid from the fluid delivery source is directed through the first catheter inner lumen to the patient vessel lumen.

41. The method of claim 33, including:
inserting at least a portion of the needle distal end and at least a portion of the first catheter distal end into the patient vessel lumen;
inserting the second catheter into the patient vessel lumen, wherein the second catheter is slidably translated longitudinally along the first catheter into the patient vessel lumen with the first catheter at least partially within the patient vessel lumen; and
removing the needle and the first catheter from the second catheter inner lumen, while maintaining the second catheter in the patient vessel lumen.

42. The method of claim 41, including
attaching a fluid delivery source to the second catheter; and
selectively directing fluid to the patient vessel lumen, wherein fluid from the fluid delivery source is directed through the second catheter inner lumen to the patient vessel lumen.

43. The method of claim 33, including fixedly joining at least a portion of the needle body and the first catheter together to be concentrically arranged to one another.

44. The method of claim 33, including slidingly joining the needle body and the first catheter together to be concentrically arranged to one another.

45. A concentric catheter device, comprising:
a needle, the needle having a needle proximal end and a needle distal end, the needle proximal end and the needle distal end being longitudinally spaced apart by an elongate needle body, the needle having a needle outer wall, the needle distal end having a needle tip;
a first catheter, the first catheter having a first catheter proximal end and a first catheter distal end, the first catheter proximal end and the first catheter distal end being longitudinally spaced apart by an elongate first catheter body, the first catheter having a first catheter outer wall, the first catheter having a first catheter inner lumen, the first catheter distal end having a first catheter open tip, the first catheter inner lumen extending between the first catheter proximal end and the first catheter open tip, at least a portion of the first catheter inner lumen being larger in diameter than the needle outer wall; and a second catheter, the second catheter having a second catheter proximal end and a second catheter distal end, the second catheter proximal end and the second catheter distal end being longitudinally spaced apart by an elongate second catheter body, the second catheter having a second catheter outer wall, the second catheter having a second catheter inner lumen, the second catheter distal end having a second catheter open tip, the second catheter inner lumen extending between the second catheter proximal end and the second catheter open tip, the second catheter inner lumen being larger in diameter than the first catheter outer wall, the second catheter proximal end being capable of being attached to a fluid source;
wherein the second catheter is slidingly joined to the first catheter,
wherein the needle and the first catheter are joined together to be concentrically arranged relative to one another, and
wherein the needle tip and at least a portion of the needle distal end longitudinally extend distally from the first catheter open tip.

46. The concentric catheter device of claim 45, including a first connector, the first connector having a first connector inner lumen and a first connector outer wall, the first connector inner lumen being larger in diameter than at least one of the first catheter outer wall and the needle body, the first connector outer wall having a first connector locking mechanism, the first connector selectively accepting at least one of the first catheter and the needle.

47. The concentric catheter device of claim 45, including a second connector, the second connector having a second connector inner lumen and a second connector outer wall, at least a portion of the second connector inner lumen being larger in diameter than the second catheter outer wall, at least a portion of the second connector inner lumen being larger in diameter than at least a portion of the first connector, the second connector inner lumen having a second connector locking mechanism, the second connector selectively accepting at least one of the second catheter and the first connector.

48. The concentric catheter device of claim 45, wherein when the needle is joined to the first catheter, the needle outer wall is directly laterally adjacent to the first catheter inner lumen.

49. The concentric catheter device of claim 45, wherein when the first catheter is joined to the second catheter, the first catheter outer wall is directly laterally adjacent to the second catheter inner lumen.

50. The concentric catheter device of claim 45, wherein when the needle is joined to the first catheter, the needle outer wall is directly laterally adjacent to the first catheter inner lumen, and when the combined needle and first catheter are joined to the second catheter, the needle outer wall is directly laterally adjacent to the first catheter inner lumen and the first catheter outer wall is directly laterally adjacent to the second catheter inner lumen.

51. The concentric catheter device of claim 45, wherein at least a portion of the needle body and the first catheter are fixedly joined together to be concentrically arranged to one another.

52. The concentric catheter device of claim 45, wherein the needle body and the first catheter are slidingly joined together to be concentrically arranged to one another.
